(19) United States
(12) Patent Application Publication
(10) Pub. No.: US 2021/0015471 A1
(43) Pub. Date: Jan. 21, 2021

(54) DEVICE FOR EXPOSING AND HOLDING OPEN AN OPERATING AREA

(71) Applicant: OLYMPUS WINTER & IBE GMBH, Hamburg (DE)

(72) Inventors: Michael WOLTER, Hamburg (DE); Lisa EFFE, Lübeck (DE); Lena WILKER, Hamburg (DE)

(73) Assignee: OLYMPUS WINTER & IBE GMBH, Hamburg (DE)

(21) Appl. No.: 16/980,752

(22) PCT Filed: Mar. 19, 2019
(86) PCT No.: PCT/EP2019/056891

§ 371 Date: Sep. 14, 2020

(30) Foreign Application Priority Data
Mar. 20, 2018 (DE) 10 2018 106 578.7

(51) Int. Cl. A61B 13/00; A61B 1/24

(52) U.S. Cl. CPC A61B 13/00; A61B 1/24

(57) ABSTRACT

A medical device for exposing and holding open an operating area in the region of the oral cavity of a patient, preferably a laryngo-pharyngoscope retractor, the device having a holding structure that is fixable in the head region of the patient for mounting medical instruments and tools and a spatula that is insertable into the oral cavity for fixing the tongue or other parts in the operating area and that includes a spatula blade and a handle region, characterized in that the spatula includes two or more mutually adjacent adjustment elements that are movable relative to one another.

### BACKGROUND

The invention relates to a medical device for exposing and holding open an operating area in the region of the oral cavity of a patient in accordance with the preamble of claim 1.

Conventional devices usually have a holding structure that is fixable in the head region of the patient and on which instruments and tools can be mounted, for example using clamping means, so as to assist in a surgical intervention in the mouth or throat of the patient. A spatula carried on the holding structure is usually used for fixing the tongue body. These spatulas generally have a handle in the manner of a flat rod, which is mounted linearly displaceably in a spatula guide. On the free end thereof protruding into the oral cavity, the spatula blade, a conventional spatula usually has a contact area specially adapted to the region to be contacted of the oral cavity. The contact area may for example be formed shell-like and have a rough surface structure so as to provide an optimum grip on the tissue to be contacted.

For adaptation to the anatomy of the patient and to the operating target, a number of different spatulas are available. These conventional devices comprising a selection of different spatulas include for example FK-WO TORS laryngo-pharyngoscope retractors (Olympus). When a conventional device is used, for example a retractor, it must initially be decided which spatula type is to be used and which spatula size is required. The selected spatula is inserted into the guide device provided for this purpose and is fixed to the holding structure. When the device is fixed in the head region of the patient, usually the holding structure is initially braced in the region of the upper jaw and the spatula is laid against the tongue of the patient. If the entry angle and insertion depth of the spatula are carefully adjusted, the upper jaw is subsequently tensioned against the spatula braced on the tongue. Spatulas of this type are formed rigid for sufficient bracing of the tongue.

The selection of the appropriate spatula for an operation requires a high level of experience, since not only the medical intervention to performed, but also the anatomy of the individual patient has to be taken into account. Moreover, readjustment of the spatula may also be necessary during an operation. During a medical intervention, however, the spatula cannot readily be replaced, since it is required for securely fixing the tongue or other parts in the space under examination. If it is necessary to select or adjust the spatula again, as a result of miscalculation or of a necessary readjustment, this currently involves considerable effort.

The object of the present invention is therefore to provide an improved device of the type set out at the outset that makes simple, rapid adaptation of the spatula possible during a medical intervention.

### DESCRIPTION

The object is achieved by a medical device according to the invention having the features of claim 1 and a spatula according to the invention having the features of claim 11. Advantageous embodiments are set out in the dependent claims.

The invention relates in particular to a medical device for exposing and holding open an operating area in the region of the oral cavity of a patient, preferably a laryngo-pharyngoscope retractor, the device having a holding structure that is fixable in the head region of the patient for mounting medical instruments and tools and a spatula that is insertable into the oral cavity for fixing the tongue or other parts in the operating area and that comprises a spatula blade and a handle region, characterized in that the spatula comprises two or more mutually adjacent adjustment elements that are movable relative to one another.

As a result of the use according to the invention of adjustment elements that are movable relative to one another, it is advantageously possible to adapt the size and shape of the spatula to the operating environment and/or to the patient size during continuous operation, without releasing the fixing of the tongue by the spatula. Adaptation of this type may be required in particular if, in the course of an intervention, the operating field or the instruments used change, or if there was initially a miscalculation regarding the required spatula size and shape.

The medical device for exposing and holding open an operating area in the region of the oral cavity of a patient is preferably a laryngo-pharyngoscope retractor. Retractors of this type are known to those skilled in the art, and are commercially available in a number of variants. The retractors may be used together with corresponding robot systems in transoral robotic surgery (TORS). The retractors provide space for placing a robot arm for this purpose. One or more of the instruments needed for the TORS may be fixed directly to the medical device according to the invention. As well as the components needed for tensioning the device in the oral cavity, this includes further instruments such as a suction device and a light carrier. For tensioning the device in the oral cavity, the device according to the invention comprises a spatula, an upper jaw support element, and optionally one or more cheek holders.

For fixing the above-described components, the device has a holding structure that is fixable in the head region of the patient for mounting medical instruments and tools. A holding structure of this type may be constructed in the manner of a frame from two substantially parallel cross-beams and two likewise mutually parallel longitudinal beams. Corresponding holding structures are known to those skilled in the art.

The device according to the invention further has a spatula that is insertable into the oral cavity for fixing the tongue or other parts in the operating area. The spatula is generally attached in a spatula guide, which is in turn fastened to the holding structure. In the released state, the spatula is displaceable within the holding structure in at least two dimensions, for example in the longitudinal direction of the oral cavity and in the direction of the upper jaw support element. In this way, the spatula may already be adapted to the anatomy and size of the patient for the operation. During the operation, the spatula is not displaced, but can according to the invention be changed in size and shape. In this way, medical staff do not have to make a selection among a plurality of different spatulas, since the same spatula can be used for the vast majority of interventions and individuals.

The spatula has a spatula blade and a handle region. The spatula blade forms the distal end region of the spatula, while the handle region forms the proximal end region. One face of the spatula blade, referred to herein as the spatula support face, is laid against the tongue when the device or spatula is applied. The spatula support face is thus the tissue contact face of the spatula. The handle region of the spatula is fastened to the holding structure of the device. Adjustment mechanisms, by means of which the spatula can be displaced in the axial direction (proximal/distal) or in the direction of the upper jaw support element (cranial/caudal), may act on the handle region. However, since adjustment of this type is only possible in the released state, the spatula according to the invention has further adjustment possibilities, which can even be operated when the spatula is fixing the tongue. For this purpose, the spatula according to the invention has two or more mutually adjacent adjustment elements that are movable relative to one another. By moving the adjustment elements relative to one another, the size and/or shape of the spatula can be changed. According to the invention, in particular the size or shape of the spatula in the extension thereof in a plane parallel to the contact plane is changed. These changes can be made while the holding structure is fixed in the region of the oral cavity of a patient for an operation intervention, without releasing the fixing of the tongue or other parts in the operating area by means of the spatula. So as to make gentle readjustment of the spatula possible, the adjustment elements described herein and the spatula support face are preferably formed to be atraumatic. In this way, the change in the size and/or shape can be made particularly gently and without injuring the tongue.

A movement of the adjustment elements relative to one another leads to adjacent adjustment elements or portions of adjacent adjustment elements being moved closer towards or further away from one another. For this purpose, the adjustment elements may be formed so as to be able to be pulled apart from or slid into one another. If the adjustment elements are moved relative to one another in an extension of the longitudinal axis of the handle region, this leads to lengthening or shortening of the spatula. If the adjustment elements are pivoted or angled out of the extension of this longitudinal axis by the relative movement, this results in a change in the shape of the spatula, namely angling of the spatula blade with respect to the handle region or widening of the spatula blade. Widening of the spatula blade brings about a change in both the shape and the size of the spatula. Herein, the term “size” denotes the area defined by the periphery of the spatula. The relative movement of the adjustment elements can thus be a rotational or pivot movement or a linear movement, preferably a linear movement in an extension of the longitudinal axis of the handle region.

While in some embodiments the adjustment elements according to the invention cause the spatula blade and the handle region to be movable relative to one another, other embodiments of the invention relate to use of the adjustment elements to widen the spatula blade. It will be appreciated that these two alternatives can also be combined with one another. Thus, a spatula according to the invention may comprise both adjustment elements for widening the spatula blade and adjustment elements for introducing movability of the spatula blade relative to the handle region.

Depending on the desired movability, the adjustment elements are held against one another in a different manner. Those skilled in the art are capable of determining suitable fastening options in each case. Examples are described herein. Thus, those skilled in the art will provide different fastening options for adjustment elements that are pivotable with respect to one another than for adjustment elements that can be slid apart and together linearly. The shape of the adjustment elements is also adapted to the desired degrees of freedom of movement in each case. Moreover, depending on the desired movability, different actuating elements may be provided. Corresponding examples are described elsewhere herein.

In a preferred embodiment, in which the spatula blade and handle region are movable relative to one another, the spatula blade is axially displaceable relative to the handle region in the extension of the longitudinal axis of the handle region. The spatula blade can be slid proximally to shorten the spatula or distally to lengthen the spatula, as required.

In an alternative embodiment, in which the spatula blade and handle region are movable relative to one another, the spatula blade can be angled out of the extension of the longitudinal axis of the handle region in a plane parallel to the contact plane. The most caudal, tongue-side part of the spatula support face is positioned in the contact plane. Moreover, in general, the handle region is also positioned in the contact plane. The contact plane thus forms, in portions, the transition from the spatula to the tongue surface. Since the spatula support face, and thus also the spatula blade, may be formed convex so as to be atraumatic, the spatula blade generally bulges, at least in the distal end region thereof, out of the contact plane. The spatula blade and handle region are movable in a parallel plane, the parallel plane being arranged cranially from the contact plane, in other words on the oral cavity side from the contact plane.

So as to make the spatula blade and handle region movable relative to one another, adjustment elements may be arranged between the spatula blade and the handle region. Alternatively or in addition, the spatula blade and/or the handle region may themselves be adjustment elements. Thus, in a very simple embodiment, the spatula may merely consist of a spatula blade, a part that forms the handle region, and a connection system. In this embodiment, the connection system makes it possible for the spatula blade to pivot out of the extension of the longitudinal axis of the handle region and thus for the spatula blade to be angled out of the extension of the longitudinal axis. However, even finer adjustment of the spatula is possible if one or more further adjustment elements are arranged between the spatula blade and the handle region.

As described, the adjustment elements may each be pivotable with respect to the adjacent adjustment element(s) in a plane parallel to the contact plane. For this purpose, it is preferred for each adjustment element to have an engagement portion and a receiving portion, the engagement portion being inserted, at least in portions, into the receiving portion of an adjacent adjustment element. Analogously to the principle described in US 2003/0184086 A1 for a round shower hose, the flat elements described herein that engage in one another are capable of providing the pivotability of the spatula blade out of the extension of the longitudinal axis of the handle region. In this context, the engagement portion preferably has a circle sector shape at the distal end thereof, while the receiving portion has a complementary circle sector shape in the interior thereof. The two circle sector shapes may slide along on one another in a substantially lateral direction.

So as not to detract from the fixing performance of the spatula, the movability according to the invention of the spatula is limited to movements in the parallel plane. Movability out of the plane in the direction of the oral cavity is undesirable. Therefore, according to the invention, pivoting out of the parallel plane can be prevented, in all embodiments described herein, by corresponding tongue-side and/or oral-cavity-side delimiting elements.

In addition or alternatively, according to the invention, the width of the spatula blade may be changeable. For this purpose, the spatula blade has two or more adjustment elements. The spatula blade may also consist of the adjustment elements. The adjustment elements are distinguished in that they can be moved, at least in portions, out of the longitudinal axis of the spatula in a radial direction. In this way, the spatula blade width can be increased. The adjustment elements preferably each extend longitudinally from the proximal end of the spatula blade to the distal end of the spatula blade. They may be arranged in a rail and be displaceable radially as a whole in the manner of a curtain. Alternatively, the spatula blade may comprise two or more adjustment elements that are movable relative to one another in the manner of a fan (in the manner of a brise fan). In this embodiment, the adjustment elements are jointly fastened to the proximal end of the spatula blade, above one another or tightly alongside one another, and can be pivoted apart at the distal ends thereof. So as to prevent tongue tissue from being caught when the adjustment elements are slid together, the adjustment elements may be formed wide enough that they continue to overlap even when slid apart.

For moving the adjustment elements, in particular for sliding them in an axial direction and/or angling them out of the extension of the longitudinal axis, the spatula generally comprises one or more actuation devices. Suitable actuation devices are routine for those skilled in the art, and comprise for example cable pulls, screw connections and the like. A suitable example of a means for axial displacement is shown in FIG. 2. However, it is also conceivable for the adjustment elements and/or the spatula blade to be slid on the tongue manually.

A related aspect of the invention relates to a spatula for use in a medical device for exposing and holding open an operating area in the region of the oral cavity of a patient, the spatula comprising a spatula blade and a handle region, characterized in that the spatula comprises two or more mutually adjacent adjustment elements that are movable relative to one another.

### BRIEF DESCRIPTION OF THE DRAWINGS

Example embodiments of the invention are schematically shown in the drawings, in which:

FIG. 1 is a schematic side view of a device according to the invention,

FIG. 2 is a schematic plan view of the holding structure of FIG. 1,

FIG. 3 is a schematic plan view of a spatula according to the invention, the spatula blade of which is axially displaceable,

FIG. 4 is a schematic plan view of a spatula according to the invention, the spatula blade of which is axially displaceable and that has a device for axial displacement,

FIG. 5 is a schematic side view of the spatula according to the invention from FIG. 4,

FIG. 6 is a schematic plan view of a spatula according to the invention, the spatula blade of which can be pivoted radially or angled out of the extension of the longitudinal axis of the handle region,

FIG. 7 is a schematic plan view of an alternative spatula according to the invention, the spatula blade of which can be angled out of the extension of the longitudinal axis of the handle region,

FIG. 8 is a schematic plan view of a further, alternative spatula according to the invention, the spatula blade of which can be angled out of the extension of the longitudinal axis of the handle region,

FIG. 9 is a schematic drawing of a spatula according to the invention, the spatula blade of which can be widened, and

FIG. 10 is a schematic plan view of a spatula according to the invention, the spatula blade of which can be widened, adjustment elements foldable in the manner of a fan being provided in the spatula blade for this purpose, which can be folded open (FIG. 10a) and closed (FIG. 10b).

### EXAMPLE EMBODIMENTS

Further advantages, characteristics and features of the present invention will be apparent from the following detailed described of example embodiments with reference to the accompanying drawings. However, the invention is not limited to these example embodiments.

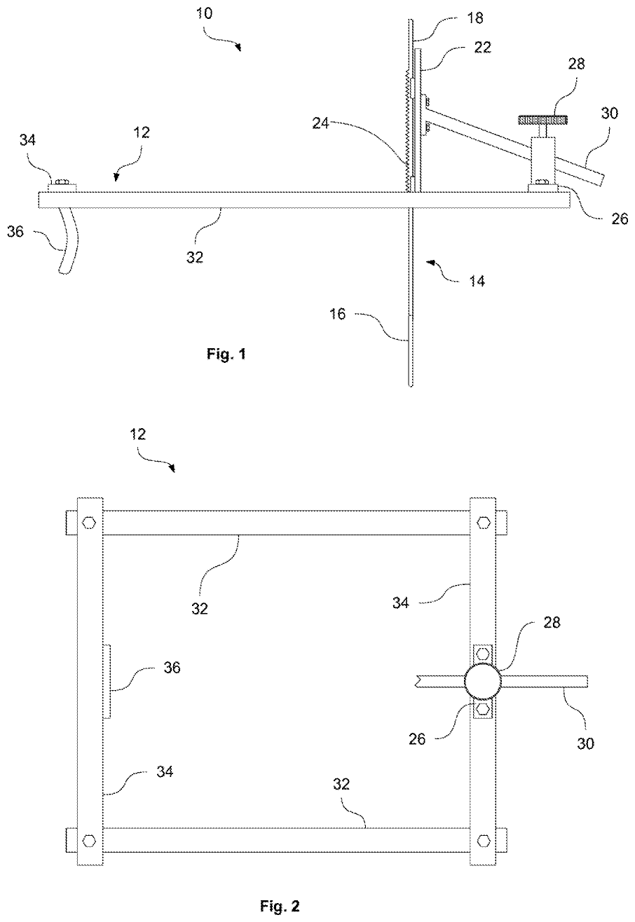

FIG. 1 shows, highly schematically, a device 10 according to the invention comprising a holding structure 12 that carries a spatula 14 that is guided height-adjustably in a spatula guide 22. As the plan view of the holding structure 12 in FIG. 2 schematically illustrates, the holding structure 12 may in particular be formed frame-like, comprising two substantially mutually parallel longitudinal beams 32, which brace two likewise approximately mutually parallel transverse beams 34. FIG. 2 is a plan view of FIG. 1, with the spatula guide 22 omitted for simplicity of the drawing.

As is shown in FIG. 1, the spatula guide 22 that carries the spatula 14 longitudinally displaceably may be fastened to a positioning element 30—in this case configured in the manner of a rod—so as to provide further options for orienting the spatula 14 relative to the holding structure 12. The positioning element 30 is also referred to as a retractor rod. In the present example, the positioning element 30 is mounted on a fastening block 26 mounted on the holding structure 12. The positioning element 30 may for example be mounted longitudinally displaceably and/or pivotably on the fastening block 30.

In the present example embodiment, the intention is that the positioning element 30 is guided longitudinally displaceably on the fastening block 26 by means of a manually actuable drive unit. For manual longitudinal displacement of the positioning element 30, a rotary control 28, for example in the form of a knurled screw, may be provided. The rotary control 28 may cooperate with the drive unit that longitudinally displaces the positioning element 30. In this context, the intention is that in the event of a rotation at the rotary control 28 the positioning element 30 is displaced in a distal or proximal direction, so as thus to move the spatula guide 22, along with the spatula 14 mounted thereon, in a distal or proximal direction relative to the fastening block 26. In addition, a locking device (not shown), arranged for example on the fastening block 26, may be provided, and fixes the positioning element 30 with respect to the fastening block 26 so as to prevent unintended displacement of the positioning element 30 after adjustment.

For fixing the device 10 in the head region of the patient, an upper jaw support element 36, which is laid against the upper jaw of a patient, may be provided on the holding structure 12. In the example shown, the upper jaw support element 36 is fastened to a first transverse beam 34 arranged distally on the holding structure 12, and the fastening block 26 that carries the positioning element 30 with the spatula guide 22 fastened thereto is arranged on a second transverse beam 34 arranged proximally on the holding structure 12. For exposing and holding open the operating area in the region of the oral cavity of the patient, the upper jaw support element 36 can be positioned against the upper jaw of the patient, and a spatula support face 38 of the spatula 14, arranged on an end region of the spatula 14, the spatula blade 16, that is insertable into the oral cavity of the patient, can be positioned against the tongue body. When the positioning element 30 is displaced in a proximal direction, a tension between the upper jaw support element 36 and the spatula 14 is generated, and serves to open the oral cavity and to hold it open. In this context, the spatula 14 presses the tongue body of the patient against the tongue base, for example. In this use position of the device 10, which fixes the upper jaw open with respect to the lower jaw, free space is created between the longitudinal beam 32 and the cross-beam 34, and gives the operator easy access to the operating area in the oral cavity of the patient. Cheek hooks (not shown here), which spread the lateral mouth open edges apart, may further be arranged on the holding structure 12 on the longitudinal beams 32, which are arranged parallel opposite one another.

For positioning the spatula 14 in the oral cavity of the patient, a handle region 18 of the spatula 14 is carried in a height-adjustable, longitudinally displaceable manner in the spatula guide 22. For locking the spatula 14 at a particular penetration depth in the oral cavity, a locking device (not shown here) may be provided on the spatula guide 22. Example locking devices are described for example in DE 10 2017 120 245.5.

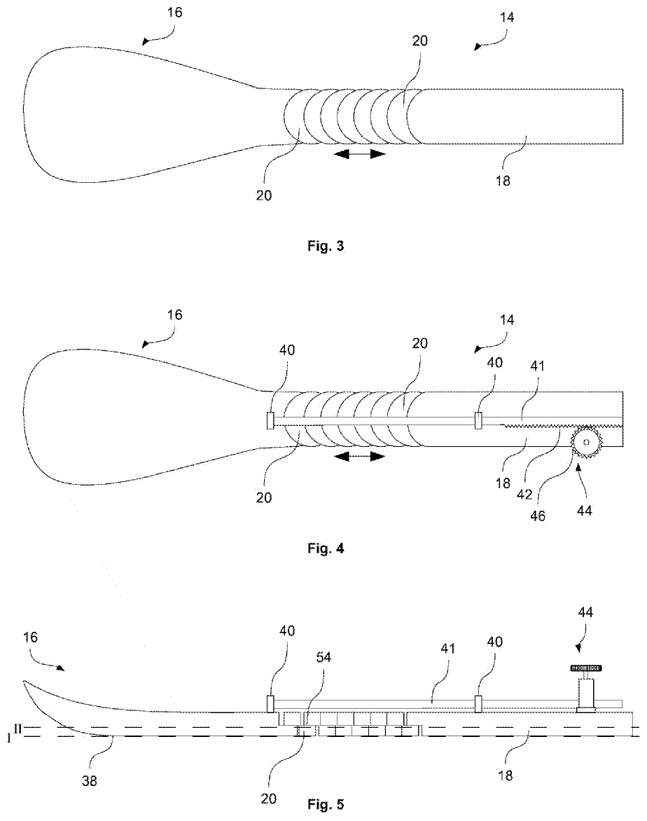

FIG. 3 is a schematic plan view of a spatula 14 according to the invention, the spatula blade 16 of which is axially displaceable. The corresponding movement and operating element for axial displacement is not shown here, but is shown by way of example in FIGS. 4 and 5. The spatula 14 has seven adjusting elements 20 between the spatula blade 16 and the handle region 18. The adjustment elements 20 are fastened to one another in such a way that they can be pulled apart from and slid back into one another axially. For this purpose, connections in which a pin-like element can be displaced within a channel on the adjacent adjustment element, in other words rail-like connecting elements, are conceivable, for example. On the tongue side, the spatula blade 18 has a spatula support face 38, which is positioned on the tongue when the device 10 is fixed.

FIG. 4 is a schematic plan view of a spatula 14 according to the invention, the spatula blade 16 of which is axially displaceable and which has a means for axially displacing the spatula blade 16 with respect to the handle region 18. With the exception of the means shown here, the spatula 14 shown basically corresponds to the spatula 14 shown in FIG. 3. The means for axially displacing the spatula blade 16 comprises a movement element 41, which is fixed to a fastening element 40 on the spatula blade 16 and moreover can slide through a further fastening element 40 on the handle region 18. When the gearwheel 46, which is part of the drive unit 44, is actuated, the movement element 41 is axially displaced under forced guidance. As is schematically indicated in FIG. 4, it may be provided that an external toothing of the gearwheel 46 engages in a toothing of a drive face 42 of the movement element 40, and thus provides axial displacement of the spatula blade 16 with respect to the handle region when the gearwheel rotates.

FIG. 5 is a schematic side view of the spatula according to the invention from FIG. 4. It can be seen that the drive unit 44 has a rotary control for actuating the gearwheel 46 (not shown). Actuating the movement element 41 brings about an axial displacement of the spatula blade 16 with respect to the handle region 18. In this context, the adjustment elements 20 are slid axially apart or together in a parallel plane II. The parallel plane II is arranged parallel to the contact plane I. In the embodiment shown here, the adjustment elements 20 each have an oral-cavity-side contact face 54 in the proximal end region thereof. The proximally adjacent adjustment element is slid onto the contact face 54.

FIG. 6 is a schematic plan view of a spatula 14 according to the invention, the spatula blade 16 of which is radially pivotable or can be angled out of the extension of the longitudinal axis of the handle region 18. A plurality of adjustment elements 20 are arranged between the spatula blade 16 and the handle region 18, and can likewise each be pivoted or rotated radially with respect to one another.

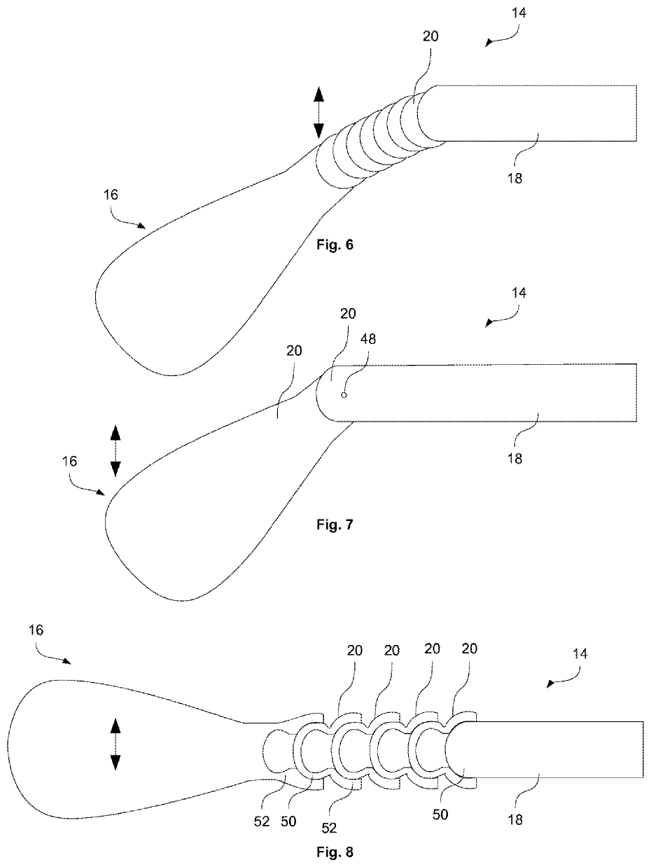

FIG. 7 is a schematic plan view of an alternative spatula 14 according to the invention, the spatula blade 16 of which can likewise be pivoted radially with respect to the handle region 18 or angled out of the extension of the longitudinal axis of the handle region 18. In the embodiment shown, the spatula blade 16 and the handle region 18 are each formed as an adjustment element 20. The proximal end region of the spatula blade 16 is mounted rotatably on the distal end region of the handle region 18 by means of a bearing 48.

FIG. 8 is a schematic plan view of a further, alternative spatula 14 according to the invention, the spatula blade 16 of which can be angled or pivoted radially out of the extension of the longitudinal axis of the handle region 18. For the pivoting, four adjustment elements 20 are arranged between the spatula blade 16 and the handle region 18. The spatula blade 16 and the handle region 18 may also be considered to be adjustment elements 20. The adjustment elements 20 each have a distal engagement portion 50 and a proximal receiving portion 52. The spatula blade 16 also has as proximal receiving portion 52, while the handle region 18 comprises an engagement portion 50 at the distal end thereof. The engagement portions 50 have a circle sector shape at the distal end thereof, and the receiving portions 52 have a complementarily shaped internal shape. The circle sector shapes are pivotable within one another parallel to the contact plane.

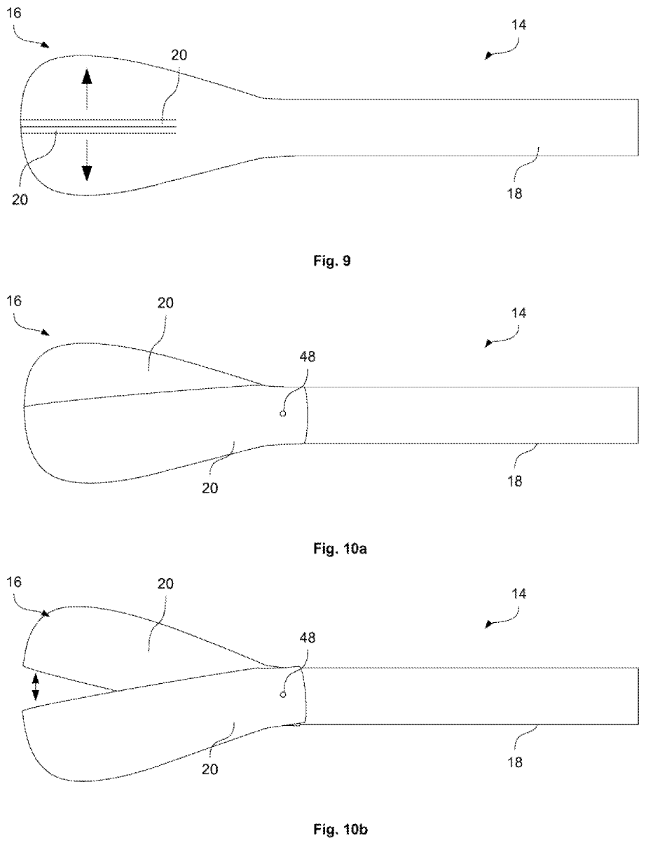

FIG. 9 is a schematic drawing of a spatula 14 according to the invention, the spatula blade 16 of which can be widened. An example technical embodiment of the principle shown is shown in FIG. 10. FIG. 10 is a schematic plan view of a spatula 14 according to the invention, the spatula blade 16 of which can be widened, two adjustment elements 20 foldable in the manner of a fan, which can be folded open (FIG. 10a) and closed (FIG. 10b), being provided in the spatula blade 16 for this purpose. It is conceivable for the spatula blade 16 to be formed from more than two fan-like adjustment elements 20, for example from three, four, five or more adjustment elements 20. The proximal end regions of the adjustment elements 20 are rotatably fastened to the distal end region of the handle region 18 via a swivel bearing 48. The adjustment elements 20 are formed to be atraumatic on the tongue-side, in a manner not shown in greater detail here.

Although the present invention has been described in detail with reference to the example embodiments, it is self-evident to a person skilled in the art that the invention is not limited to these example embodiments, numerous modifications being possible in that individual features can be omitted or different combinations of the disclosed individual features can be implemented so long as there is no departure from the scope of protection of the accompanying claims. The present disclosure includes all combinations of the disclosed individual features.

### LIST OF REFERENCE NUMERALS

10 Medical device
12 Holding structure
14 Spatula
16 Spatula blade
18 Handle region
20 Adjustment element
22 Spatula guide
24 Drive face
26 Fastening block
28 Rotary control
30 Positioning element
32 Longitudinal beam
34 Cross-beam
36 Upper jaw support element
38 Spatula support face
40 Fastening element
41 Movement element
42 Drive face
44 Drive unit
46 Gearwheel
48 Bearing
50 Engagement portion
52 Receiving portion
54 Contact face
I Contact plane
II Parallel plane

1. Medical device for exposing and holding open an operating area in the region of the oral cavity of a patient, the device having a holding structure that is fixable in the head region of the patient for mounting medical instruments and tools and a spatula that is insertable into the oral cavity for fixing the tongue or other parts in the operating area and that comprises a spatula blade and a handle region, wherein the spatula comprises two or more mutually adjacent adjustment elements that are movable relative to one another.

2. Device according to claim 1, wherein by moving the adjustment elements relative to one another, the size and/or shape of the spatula can be changed, in particular while the holding structure is fixed in the region of the oral cavity of a patient for an operation intervention, without releasing the fixing of the tongue or other parts in the operating area by means of the spatula.

3. Device according to claim 1, wherein the spatula blade and the handle region are movable relative to one another.

4. Device according to claim 1, wherein the spatula blade is axially displaceable relative to the handle region in the extension of the longitudinal axis of the handle region.

5. Device according to claim 1, wherein the spatula blade can be angled out of the extension of the longitudinal axis of the handle region in a plane (II) parallel to the contact plane (I).

6. Device according to claim 1, wherein one or more adjustment elements are arranged between the spatula blade and the handle region.

7. Device according to claim 1, wherein the adjustment elements are each pivotable relative to the adjacent adjustment element(s) in a plane (II) parallel to the contact plane, each adjustment element having an engagement portion and a receiving portion, and the engagement portion being inserted, at least in portions, into the receiving portion of an adjacent adjustment element.

8. Device according to claim 1, wherein the spatula blade and/or the handle region are adjustment elements.

9. Device according to claim 1, wherein the width of the spatula blade can be changed.

10. Device according to claim 1, wherein the spatula blade comprises two or more adjustment elements that are movable relative to one another in the manner of a fan.

11. Spatula for use in a medical device for exposing and holding open an operating area in the region of the oral cavity of a patient, the spatula comprising a spatula blade and a handle region, wherein the spatula comprises two or more mutually adjacent adjustment elements that are movable relative to one another.
